(19) United States
(12) Patent Application Publication
(10) Pub. No.: US 2010/0234791 A1
(43) Pub. Date: Sep. 16, 2010

(54) DUAL DRAINAGE PATHWAY SHUNT DEVICE

(75) Inventors: Mary G. Lynch, Atlanta, GA (US); Reay H. Brown, Atlanta, GA (US); Guido Smeets, Palo Alto, CA (US)

Correspondence Address: KNOBBE MARTENS OLSON & BEAR LLP, 2040 MAIN STREET, FOURTEENTH FLOOR, IRVINE, CA, 92614, US

(73) Assignee: Glaukos Corporation, Laguna Hills, CA (US)

(21) Appl. No.: 12/785,306

(22) Filed: May 21, 2010

Related U.S. Application Data
(62) Division of application No. 11/742,484, filed on Apr. 30, 2007.
(60) Provisional application No. 60/796,424, filed on May 1, 2006.

(51) Int. Cl. A61M 1/00

(52) U.S. Cl. 604/8

(57) ABSTRACT

A shunt is provided for the flow of aqueous humor from the anterior chamber of the eye to Schlemm's canal and to other anatomical spaces of the eye. The shunt comprises at least one lumen and optionally has at least one anchor extending from a proximal portion of the shunt to assist in placement and anchoring of the device in the correct anatomic position.

### CROSS-REFERENCE TO RELATED APPLICATIONS

This application is a divisional of U.S. application Ser. No. 11/742,484, filed on Apr. 30, 2007, which claims priority under 35 U.S.C. §119(e) to U.S. Provisional Application No. 60/796,424, filed on May 1, 2006, the disclosure of each of which is incorporated by reference herein in its entirety. This application also cross references U.S. application Ser. No. 10/899,687, filed Jul. 27, 2004, now U.S. Pat. No. 7,220,238, which is a continuation of U.S. application Ser. No. 10/222,209, filed Aug. 16, 2002, which a) claims priority under 35 U.S.C. §119(e) to U.S. Provisional Application Ser. No. 60/312,799, filed Aug. 16, 2001 and b) is a Continuation-In-Part of U.S. application Ser. No. 09/558,505, filed Apr. 26, 2000, now U.S. Pat. No. 6,450,984, which claims priority under 35 U.S.C. §119(e) to U.S. Provisional Application No. 60/131,030, filed Apr. 26, 1999, all of which are incorporated in their entirety by reference herein.

### BACKGROUND OF THE INVENTION

1. Field of the Invention

The present invention is generally directed to a surgical treatment for glaucoma, and relates more particularly to a device and method for continuously decompressing elevated intraocular pressure in eyes affected by glaucoma by diverting aqueous humor from the anterior chamber of the eye into Schlemm's canal and into the sub scleral or uveoscleral spaces.

2. Description of the Related Art

Glaucoma is a significant public health problem, because glaucoma is a major cause of blindness. The blindness that results from glaucoma involves both central and peripheral vision and has a major impact on an individual's ability to lead an independent life.

Glaucoma is an optic neuropathy (a disorder of the optic nerve) that usually occurs in the setting of an elevated intraocular pressure. The pressure within the eye increases and this is associated with changes in the appearance (“cupping”) and function (“blind spots” in the visual field) of the optic nerve. If the pressure remains high enough for a long enough period of time, total vision loss occurs. High pressure develops in an eye because of an internal fluid imbalance.

The eye is a hollow structure that contains a clear fluid called “aqueous humor.” Aqueous humor is formed in the posterior chamber of the eye by the ciliary body at a rate of about 2.5 microliters per minute. The fluid, which is made at a fairly constant rate, then passes around the lens, through the pupillary opening in the iris and into the anterior chamber of the eye. Once in the anterior chamber, the fluid drains out of the eye through two different routes. In the “uveoscleral” route, the fluid percolates between muscle fibers of the ciliary body. This route accounts for approximately ten percent of the aqueous outflow in humans. The primary pathway for aqueous outflow in humans is through the “canalicular” route that involves the trabecular meshwork and Schlemm's canal.

The trabecular meshwork and Schlemm's canal are located at the junction between the iris and the sclera. This junction or corner is called “the angle.” The trabecular meshwork is a wedge-shaped structure that runs around the circumference of the eye. It is composed of collagen beams arranged in a three-dimensional sieve-like structure. The beams are lined with a monolayer of cells called trabecular cells. The spaces between the collagen beams are filled with an extracellular substance that is produced by the trabecular cells. These cells also produce enzymes that degrade the extracellular material. Schlemm's canal is adjacent to the trabecular meshwork. The outer wall of the trabecular meshwork coincides with the inner wall of Schlemm's canal. Schlemm's canal is a tube-like structure that runs around the circumference of the cornea. In human adults, Schlemm's canal is believed to be divided by septa into a series of autonomous, dead-end canals.

The aqueous fluid travels through the spaces between the trabecular beams, across the inner wall of Schlemm's canal and into the canal, through a series of about 25 collecting channels that drain from Schlemm's canal and into the episcleral venous system. In a normal situation, aqueous production is equal to aqueous outflow and intraocular pressure remains fairly constant in the 15 to 21 mmHg range. In glaucoma, the resistance through the canalicular outflow system is abnormally high causing reduced outflow thereby increasing pressure.

In primary open angle glaucoma, which is the most common form of glaucoma, the abnormal resistance is believed to be along the outer aspect of trabecular meshwork and the inner wall of Schlemm's canal. It is believed that an abnormal metabolism of the trabecular cells leads to an excessive build up of extracellular materials or a build up of abnormally “stiff” materials in this area. Primary open angle glaucoma accounts for approximately eighty-five percent of all glaucoma. Other forms of glaucoma (such as angle closure glaucoma and secondary glaucomas) also involve decreased outflow through the canalicular pathway but the increased resistance is from other causes such as mechanical blockage, inflammatory debris, cellular blockage, etc.

With the increased resistance, the aqueous fluid builds up because it cannot exit fast enough. As the fluid builds up, the intraocular pressure (IOP) within the eye increases. The increased IOP compresses the axons in the optic nerve and also may compromise the vascular supply to the optic nerve. The optic nerve carries vision from the eye to the brain. Some optic nerves seem more susceptible to IOP than other eyes. While research is investigating ways to protect the nerve from an elevated pressure, the only therapeutic approach currently available in glaucoma is to reduce the intraocular pressure.

The clinical treatment of glaucoma is approached in a step-wise fashion. Medication often is the first treatment option. Administered either topically or orally, these medications work to either reduce aqueous production or they act to increase outflow. Currently available medications have many serious side effects including: congestive heart failure, respiratory distress, hypertension, depression, renal stones, aplastic anemia, sexual dysfunction and death. Compliance with medication is a major problem, with estimates that over half of glaucoma patients do not follow their correct dosing schedules.

When medication fails to adequately reduce the pressure, often surgical treatment is performed as a next step in glaucoma treatment. In laser trabeculoplasty, thermal energy from a laser is applied to a number of noncontiguous spots in the trabecular meshwork. It is believed that the laser energy stimulates the metabolism of the trabecular cells in some way, and changes the extracellular material in the trabecular meshwork. In approximately eighty percent of patients, aqueous outflow is enhanced and IOP decreases. However, the effect often is not long lasting and fifty percent of patients develop an elevated pressure within five years. The laser surgery is not usually repeatable. In addition, laser trabeculoplasty is not an effective treatment for primary open angle glaucoma in patients less than fifty years of age, nor is it effective for angle closure glaucoma and many secondary glaucomas. If laser trabeculoplasty does not reduce the pressure enough, then filtering surgery is performed. With filtering surgery, a hole is made in the sclera and angle region. This hole allows the aqueous fluid to leave the eye through an alternate route.

The most commonly performed filtering procedure is a trabeculectomy. In a trabeculectomy, a posterior incision is made in the conjunctiva, the transparent tissue that covers the sclera. The conjunctiva is rolled forward, exposing the sclera at the limbus. A partial thickness scleral flap is made and dissected half-thickness into the cornea. The anterior chamber is entered beneath the scleral flap and a section of deep sclera and trabecular meshwork is excised. The scleral flap is loosely sewn back into place. The conjunctival incision is tightly closed. Post-operatively, the aqueous fluid passes through the hole, beneath the scleral flap and collects in an elevated space beneath the conjunctiva. The fluid then is either absorbed through blood vessels in the conjunctiva or traverses across the conjunctiva into the tear film.

Trabeculectomy is associated with many problems. Fibroblasts that are present in the episclera proliferate and migrate and can scar down the scleral flap. Failure from scarring may occur, particularly in children and young adults. Of eyes that have an initially successful trabeculectomy, eighty percent will fail from scarring within three to five years after surgery. To minimize fibrosis, surgeons now are applying antifibrotic agents such as mitomycin C (MMC) and 5-fluorouracil (5-FU) to the scleral flap at the time of surgery. The use of these agents has increased the success rate of trabeculectomy but also has increased the prevalence of hypotony. Hypotony is a problem that develops when aqueous flows out of the eye too fast. The eye pressure drops too low (usually less than 6.0 mmHg); the structure of the eye collapses and vision decreases.

Trabeculectomy creates a pathway for aqueous fluid to escape to the surface of the eye. At the same time, it creates a pathway for bacteria that normally live on the surface of the eye and eyelids to get into the eye. If this happens, an internal eye infection can occur called endophthalmitis. Endophthalmitis often leads to permanent and profound visual loss. Endophthalmitis can occur anytime after trabeculectomy. The risk increases with the thin blebs that develop after MMC and 5-FU. Another factor that contributes to infection is the placement of a bleb. Eyes that have trabeculectomy performed inferiorly have about five times the risk of eye infection than eyes that have a superior bleb. Therefore, initial trabeculectomy is performed superiorly under the eyelid, in either the nasal or temporal quadrant.

In addition to scarring, hypotony and infection, there are other complications of trabeculectomy. The bleb can tear and lead to profound hypotony. The bleb can be irritating and can disrupt the normal tear film, leading to blurred vision. Patients with blebs generally cannot wear contact lenses. All of the complications from trabeculectomy stem from the fact that fluid is being diverted from inside the eye to the external surface of the eye.

When trabeculectomy doesn't successfully lower the eye pressure, the next surgical step often is an aqueous shunt device. An aqueous diversion device of the prior art is a silicone tube that is attached at one end to a plastic (polypropylene or other synthetic) plate. With an aqueous shunt device, an incision is made in the conjunctiva, exposing the sclera. The plastic plate is sewn to the surface of the eye posteriorly, usually over the equator. A full thickness hole is made into the eye at the limbus, usually with a needle. The tube is inserted into the eye through this hole. The external portion of the tube is covered with either donor sclera or pericardium. The conjunctiva is replaced and the incision is closed tightly.

With prior art aqueous diversion devices, aqueous drains out of the eye through the silicone tube to the surface of the eye. Deeper orbital tissues then absorb the fluid. The outside end of the tube is protected from fibroblasts and scarring by the plastic plate. Many complications are associated with aqueous shunt devices. A thickened wall of scar tissue that develops around the plastic plate offers some resistance to outflow and in many eyes limits the reduction in eye pressure. In some eyes, hypotony develops because the flow through the tube is not restricted. Many physicians tie an absorbable suture around the tube and wait for the suture to dissolve post-operatively at which time enough scar tissue has hopefully formed around the plate sufficiently to slow outflow. Some devices contain a pressure-sensitive valve within the tube, although these valves may not function properly and are a source of potential complications and failures. The surgery involves operating in the posterior orbit and many patients develop an eye muscle imbalance and double vision post-operatively. With prior art aqueous shunt devices because they are open to the surface of the eye, a pathway is created for bacteria to get into the eye and endophthalmitis can potentially occur.

The prior art includes a number of such aqueous shunt devices, such as U.S. Pat. No. 4,936,825 (providing a tubular shunt from the anterior chamber to the corneal surface for the treatment of glaucoma), U.S. Pat. No. 5,127,901 (directed to a transscleral shunt from the anterior chamber to the subconjunctival space), U.S. Pat. No. 5,180,362 (teaching a helical steel implant that is placed to provide drainage from the anterior chamber to the subconjunctival space), and U.S. Pat. No. 5,433,701 (generally teaching shunting from the anterior chamber to the scleral or conjunctival spaces).

In addition to the prior art aqueous shunt devices described above, other prior art devices for glaucoma surgery have used setons, or other porous, wick-like components to divert and convey excess aqueous from the anterior chamber to the exterior ocular surface. Examples include U.S. Pat. Nos. 4,634,418 and 4,787,885 (teaching the surgical treatment of glaucoma using an implant that consists of a triangular seton (wick)), and U.S. Pat. No. 4,946,436, (teaching the use of a porous device to shunt anterior chamber to subscleral space). Also see U.S. published patent application US20040015140A 1 also showing subscleral placement.

Subscleral/uveoscleral placement, including placement into the subscleral supra-ciliary space, has also been attempted. These procedures also only rely on one drainage pathway and do not teach placement in Schlemm's canal.

Some prior art references for glaucoma management have been directed at Schlemm's canal, but these have not involved the placement of long-term, indwelling shunts. U.S. Pat. No. 5,360,399 teaches the temporary placement of a plastic or steel tube with preformed curvature in Schlemm's canal with injection of a viscous material through the tube to hydraulically expand and hydrodissect the trabecular meshwork. The tube is removed from the canal following injection. Because the tube is directed outwardly from the eye for injection access, the intersection of the outflow element with the preformed curved element within Schlemm's canal is at about a 90 degree angle relative to the plane of the curvature, and 180 degrees away from the anterior chamber. Therefore, at no time does any portion of the '399 device communicate with the anterior chamber. Furthermore, relative to that portion within Schlemm's canal, this tube has a larger diameter injection cuff element, which serves as an adapter for irrigation. Therefore, this device is not adapted for shunting aqueous between the anterior chamber and Schlemm's canal.

Most of the problems that have developed with current glaucoma treatment devices and procedures have occurred because aqueous fluid is drained from inside of the eye to the surface of the eye. A need exists, then, for a more physiologic system to enhance the drainage of aqueous fluid from the anterior chamber into Schlemm's canal. The intention of the present invention is to use the existing physiologic canalicular and uveoscleral pathways for drainage of the excess in intra-ocular fluids.

In the vast majority of glaucoma patients, the resistance problem lies between Schlemm's canal and the anterior chamber. Without any prior surgical intervention, the canal itself, the collecting channels and the episcleral venous system all are intact. Enhancing aqueous flow directly into Schlemm's canal would minimize the scarring that usually occurs with an external filtration procedure since the internal angle region is populated with a single line of non-proliferating trabecular cells. Enhancing aqueous flow directly into Schlemm's canal would minimize hypotony since the canal is part of the normal outflow system and is biologically engineered to handle the normal volume of aqueous humor. Additionally, enhancing aqueous flow directly into Schlemm's canal would eliminate complications such as endophthalmitis and leaks.

### SUMMARY OF THE INVENTION

While the use of Schlemm's canal as a drainage pathway for glaucoma patients appears feasible, clinical studies have shown that a single device draining from the anterior chamber solely into Schlemm's canal may not provide adequate drainage in some patients to reduce pressures to acceptable levels. Further, when pressures are decreased after surgery, in some patients they may begin to increase again at an unpredictable time in the future. What appears necessary from anecdotal experience is the addition of an alternative drainage pathway to combine with use of Schlemm's canal to provide a plurality of routes to control intraocular pressure.

Accordingly, one aspect of the present invention is directed to providing method for the treatment of glaucoma in which one or more shunts are placed to facilitates both the normal physiologic pathway for drainage of aqueous humor from the anterior chamber of the eye into Schlemm's canal, and into other anatomic spaces in the eye, such as the scleral, suprachoroidal (or uveoscleral), or subconjunctival spaces.

Another aspect of the present invention involves a method for reducing intraocular pressure within an eye. A first outflow route is established for draining aqueous humor from the anterior chamber to Schlemm's canal by inserting an implantable member through the trabecular meshwork. A second outflow route is established for draining aqueous humor from the anterior chamber to the suprachoroidal space. The second outflow route is established by inserting an implantable member in tissue proximate the suprachoroidal space such that a distal end of the implantable member drains aqueous humor to the suprachoroidal space.

An apparatus for reducing intraocular pressure is disclosed in accordance with some embodiments of the present invention. In some embodiments, the apparatus comprises an implant having a proximal portion and a distal portion with a lumen extending therebetween, the lumen having a sufficient length to drain aqueous humor from the anterior chamber of an eye into the suprachoroidal space of the eye when implanted, the implant having an anchor portion disposed at the proximal portion.

In some embodiments, the anchor potion can be configured to engage adjacent tissue when implanted. In some embodiments, the anchor potion can comprise a surface which can facilitate growth of cells. In some embodiments, the anchor portion can be spaced from the distal portion. In some embodiments, the anchor portion can be located outside the anterior chamber when implanted. In some embodiments, the anchor portion can be spaced from a proximal end of the proximal portion. In some embodiments, the length of the lumen can be between about 4 mm to about 6 mm. In some embodiments, the anchor portion can comprise at least one groove formed on an exterior surface of the implant. In some embodiments, the apparatus can additionally comprise a valve arranged to inhibit flow through the lumen in at least one direction.

A method for reducing intraocular pressure within an eye is disclosed in accordance with another embodiment of the present invention. In some embodiments, the method comprises: establishing a first outflow route for draining aqueous humor from the anterior chamber to Schlemm's canal, wherein said establishing said first outflow route involves inserting an implantable member through the trabecular meshwork; and establishing a second outflow route for draining aqueous humor from the anterior chamber to the suprachoroidal space, wherein said establishing said second outflow route involves inserting an implantable member in tissue proximate the suprachoroidal space such that a distal end of the implantable member drains aqueous humor to the suprachoroidal space.

In some embodiments, the implantable members forming the outflow routes can be connected to each other. In some embodiments, the first outflow route and the second outflow route can partially overlap. In some embodiments, the first outflow route can be established before the second outflow route. In some embodiments, the method can additionally involve inhibiting flow through at least one of the first and second outflow routes.

A method for reducing intraocular pressure is disclosed in accordance with another embodiment of the present invention. In some embodiments, the method comprises: positioning an implant to drain aqueous humor from the anterior chamber of an eye to the suprachoroidal space of the eye, such that a proximal end of a proximal portion of the implant resides in the anterior chamber of the eye; and anchoring the proximal portion of the implant in tissue adjacent the proximal portion of the implant.

In some embodiments, the anchoring can comprise providing a surface on the implant which facilitates growth of cells. In some embodiments, the anchoring can comprise anchoring at a location outside the anterior chamber of the eye. In some embodiments, the anchoring can comprise anchoring at a location spaced from a distal end of the implant. additionally comprising conducting aqueous humor though a lumen having a length of about 4 mm to about 6 mm. In some embodiments, the method can additionally involve inhibiting flow through the implant in at least one flow direction.

### BRIEF DESCRIPTION OF THE DRAWINGS

FIG. 1A is an illustration showing an overhead perspective view of one embodiment of the present invention, in which a shunt is comprised of tubular elements extending from the anterior chamber in the eye bi-directionally within Schlemm's canal.

FIG. 1B is an overhead view of a longitudinal cross section of the embodiment shown in FIG. 1A, detailing the internal communication between the lumens of the tubular elements comprising the present device.

FIG. 1C is an illustration showing an overhead perspective view of one embodiment of the present invention, in which a shunt is comprised of mesh tubular elements extending from the anterior chamber of the eye bi-directionally within Schlemm's canal.

FIG. 1D is an illustration showing an overhead perspective view of one embodiment of the present invention, in which a shunt is comprised of solid, porous elements extending from the anterior chamber of the eye bi-directionally within Schlemm's canal.

FIG. 1E is an overhead perspective view of another embodiment of the present invention, with phantom lines detailing two lumens within the present device.

FIG. 2 is an illustration showing another embodiment of the present invention, in which a shunt is comprised of perforated tubular elements and with an angulated terminal aspect of the proximal portion.

FIG. 3A is an illustration showing a perspective of another embodiment of the present invention in which a shunt is comprised of elements that are partially tubular and partially open in their configuration.

FIG. 3B is an illustration showing a top view of the embodiment shown in FIG. 3A, with phantom lines detailing the internal communication of the device.

FIG. 3C is an illustration showing a side view from the proximal end of the embodiment of FIG. 3A.

FIG. 3D is an illustration showing a perspective of another embodiment of the present invention in which a shunt is comprised of elements that are partially open and trough-like in configuration.

FIG. 4 is an illustration showing another embodiment of the present invention, in which a shunt is comprised of distal elements having wicking extensions at their terminal ends, and in which the proximal portion has a sealed, blunted tip with a portal continuous with the lumen of the proximal portion, oriented to face away from the iris when the device is implanted in the anterior chamber.

FIG. 5A is an illustration showing another embodiment of the shunt in which a portion of the device enters Schlemm's canal in only one direction and shunts fluid in a non-linear path from the anterior chamber.

FIG. 5B is an illustration showing an additional embodiment of the shunt in which the entire shunt is placed within Schlemm's canal but contains a fenestration to maintain fluid egress of aqueous humor from the anterior chamber to Schlemm's canal.

FIG. 5C is an illustration showing a side view of one embodiment of the present invention, in which a shunt is comprised of tubular elements, with a proximal portion extending towards the anterior chamber that is shorter relative to the distal portions which extend bi-directionally within Schlemm's canal.

FIG. 5D is an illustration showing an additional embodiment of the shunt comprised of a partially open trough-like element which is placed within Schlemm's canal but contains a portal to maintain fluid egress of aqueous humor from the anterior chamber to Schlemm's canal.

FIG. 5E is an illustration showing an additional embodiment of the shunt comprised of a solid, but porous, wick-like element which is placed within Schlemm's canal

FIG. 6A is a cross-sectional illustration showing the anatomic relationships of the location of an exemplary embodiment of the shunt into both the anterior chamber of the eye and Schlemm's canal.

FIG. 6B is a cross-sectional illustration showing the anatomic relationships of the surgical placement of an exemplary embodiment of the shunt.

FIG. 6C is a cross-sectional illustration showing the anatomic relationships of the surgical placement of another exemplary embodiment of the shunt in which the proximal portion has an angulated terminal aspect with a sealed, blunted tip with a portal continuous with the lumen of the proximal portion, oriented to face away from the iris when the device is implanted in Schlemm's canal.

FIG. 7A is a cross-sectional illustration showing the anatomic relationships of the surgical placement of an exemplary embodiment of the shunt showing the proximal portion of the device and a barb-shaped anchor extending toward the iris.

FIG. 7B is a cross-sectional illustration showing the anatomic relationships of the surgical placement of another exemplary embodiment of the shunt showing the proximal portion of the device having an annular or circumferential anchor thereon.

FIG. 8A shows one embodiment of the device having a bi-directional distal portion and an anchor on the proximal portion extending circumferentially thereon.

FIG. 8B shows another embodiment of the device having a bi-directional distal portion and an anchor on the proximal portion extending medially toward the location of the iris when implanted.

FIG. 8C shows another embodiment of the device having a bi-directional distal portion and an anchor on the proximal portion extending laterally on each side of the device when implanted.

FIG. 9 shows another embodiment having a bi-directional distal portion and an anchor on the proximal portion extending circumferentially thereon in a barbed or cone shape to facilitate introduction into the anterior chamber and to inhibit removal therefrom.

FIG. 10 shows another embodiment having a tapered proximal portion with screw threads.

FIG. 11 shows an embodiment having three discrete lumens connecting the anterior chamber from the proximal tri-luminal end, bi-directionally into Schlemm's canal, and into other eye spaces, such as the suprachoroidal (uveoscleral), the scleral, and subconjunctival spaces.

FIG. 12 shows an embodiment having three discrete lumens draining aqueous fluid from the anterior chamber into Schlemm's canal and to other spaces in the eye.

FIG. 13A shows an embodiment having two unequal length distal arms intended to drain aqueous fluid from the anterior chamber into Schlemm's canal and to other spaces of the eye.

FIG. 13B shows an embodiment having a single lumen which bifurcates into two lumens intended to drain aqueous fluid from the anterior chamber into Schlemm's canal and to other spaces of the eye.

FIG. 13C shows an embodiment having two different diameter distal arms intended to drain aqueous fluid from the anterior chamber into Schlemm's canal and to other spaces of the eye.

FIG. 14A is an illustration showing an overhead perspective view of one embodiment of the shunt, in which the shunt is comprised of tubular elements extending from the anterior chamber in the eye bi-directionally within Schlemm's canal and the sclera.

FIG. 14B is an overhead view of a longitudinal cross section of the embodiment of the present shunt shown in FIG. 14A, detailing the internal communication between the lumens of the tubular elements comprising the shunt.

FIG. 14C is an illustration showing an overhead perspective view of one embodiment of the shunt, in which the shunt comprises mesh tubular elements extending from the anterior chamber in the eye bi-directionally within Schlemm's canal and a third element extending into other spaces of the eye.

FIG. 14D is an illustration showing an overhead perspective view of one embodiment of the shunt, in which the shunt is comprised of solid, porous elements extending from the anterior chamber of the eye bi-directionally within Schlemm's canal and within other spaces of the eye.

FIG. 15 is a cross-sectional illustration showing the anatomic relationships of the location of an exemplary embodiment of the shunt into the anterior chamber of the eye, Schlemm's canal and other places in the eye.

FIG. 16 is a cross-sectional illustration showing a surgical pathway for implantation into the anterior chamber, Schlemm's canal and other places of the eye.

FIG. 17 is another cross-sectional illustration showing a surgical pathway for implantation into the anterior chamber, Schlemm's canal and other places of the eye.

### DETAILED DESCRIPTION OF THE PREFERRED EMBODIMENT

The present invention provides aqueous humor shunt devices to divert aqueous humor in the eye from both the anterior chamber into Schlemm's canal and a second anatomical space in the eye. The shunt devices can comprise a first distal portion having a terminal aspect sized and shaped to be received within a portion of Schlemm's canal, a second distal portion sized and shaped to be received with a second anatomical space in the eye and a proximal portion having at least one terminal aspect sized and shaped to be received within the anterior chamber of the eye, wherein the device permits fluid communication between the proximal portion in the anterior chamber to the distal portions in Schlemm's canal and the second anatomical space. Fluid communication can be facilitated by an aqueous humor directing channel in either the proximal or distal portions, as described below. Fluid communication can also be facilitated by the use of a seton, or other porous, wick-like components at the solid proximal or distal portions of the device, for example.

The present invention also provides embodiments of an inventive shunt comprising a body of biocompatible material of a size and shape adapted to be at least partially circumferentially received within a portion of Schlemm's canal to divert aqueous humor from the anterior chamber of the human eye to and within Schlemm's canal, and wherein the body facilitates the passage of aqueous humor from the anterior chamber into Schlemm's canal. This embodiment of the device can be produced without the proximal portion of the previous embodiment extending into the anterior chamber. This embodiment can also be used to facilitate drainage from Schlemm's canal. An aqueous humor directing channel can facilitate the passage of aqueous humor from the anterior chamber into Schlemm's canal. Fluid communication can also be facilitated by the use of a seton, or other porous, wick-like components at the solid proximal or distal portions of the device, for example.

The invention contemplates many different configurations for an aqueous humor directing channel, provided that each assists in channeling aqueous humor from the anterior chamber to Schlemm's canal, such as by providing a lumen, trough, wick or capillary action. For example, the aqueous humor directing channel can be a fully enclosed lumen, a partially enclosed lumen, or a trough-like channel that is at least partially open. The invention contemplates that a solid monofilament or braided polymer, such as Proline (polypropylene), can be inserted into Schlemm's canal to provide a wicking or stenting function to facilitate the passage of aqueous humor from the anterior chamber to Schlemm's canal. Such a wicking or stenting extension can also be grooved or fluted along any portion of the length thereof, so as to be multi-angular or star-shaped in cross-section having multiple channels for fluid flow. The devices can be constructed of a solid, matrix, mesh, fenestrated, or porous material, or combinations thereof.

Traditional glaucoma teaching states that Schlemm's canal in an adult is divided by septa into separate canals, rendering the complete passage of a suture impossible. Preliminary studies on adult human eye bank eyes have shown that Schlemm's canal is, indeed, patent. A suture can be passed through the entire circumference of the canal. It has not been heretofore determined that Schlemm's canal is patent throughout its circumference in normal adult individuals, as opposed to being divided by septae into multiple dead end canals. The invention utilizes this knowledge to create and maintain the natural physiologic egress of aqueous humor from the anterior chamber to Schlemm's canal and to the collecting channels.

The present invention also provides methods of use of the shunt devices. One embodiment of the present invention is directed to a surgical method to divert aqueous humor from the anterior chamber of the eye into Schlemm's canal with a device that is implanted to extend from within the anterior chamber to Schlemm's canal and from the anterior chamber to a second anatomical space or from Schlemm's canal to a second anatomical space. The portion of the device extending into Schlemm's canal can be fashioned from a flexible material, such as silicone, capable of being received within a portion of the radius, curvature, and diameter of Schlemm's canal. The external diameter of the proximal portion can be about 0.01 mm to 0.5 mm, or about 0.3 mm. Preliminary studies indicate a preferred diameter for the proximal portion to be about 0.23 mm to about 0.28 mm, or preferably about 0.23 mm to about 0.26 mm. All or parts of the device may be solid, porous, tubular, trough-like, fenestrated, or pre-curved. The portion of the device extending into the second anatomical space is sized to fit the second anatomical space.

The second anatomical space can be any suitable space for implantation of the shunt. This space, for example, can be within the sclera or within the uveoscleral spaces. Using the scleral route involves implantation of the device inside the sclera at a sufficient distance from the conjunctiva to provide resistance to flow and to maintain sufficient tissue above the device to prevent infiltration of bacteria or other contaminants.

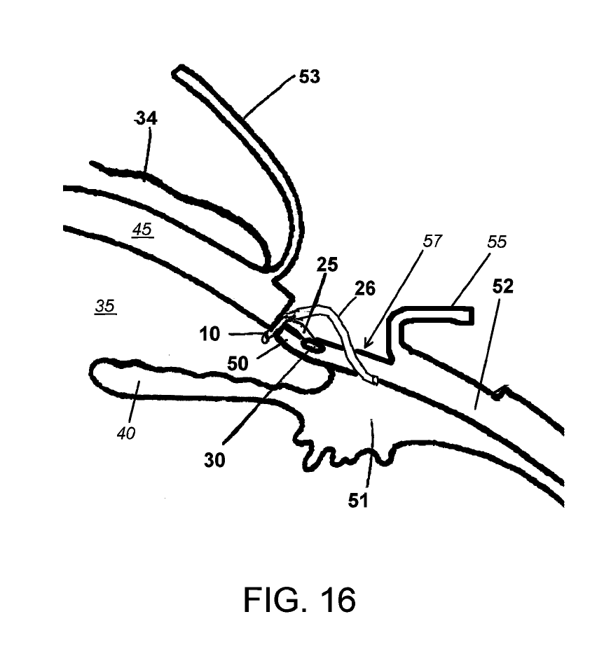

The uveoscleral route through the supra-choroidal space is less clear with regard to anatomy and physiologic significance, but probably accounts for 10-20% of aqueous outflow in the normal human eye. As with the canalicular route, the uveoscleral pathway begins in the anterior chamber angle. The aqueous is absorbed by portions of the peripheral iris, the ciliary body and probably the trabecular meshwork, from whence it passes posteriorly through the longitudinal muscle of the ciliary body to the suprachoroidal space (between the choroids and sclera). Aqueous in the suprachoroidal space may pass as far posteriorly as the optic nerve and leave the eye through a variety of emissaria around nerves and vessels in the sclera. The uveoscleral route involves fitting a distal end of the device into the potential space between the ciliary body 51 and the sclera 52 as shown in FIG. 16.

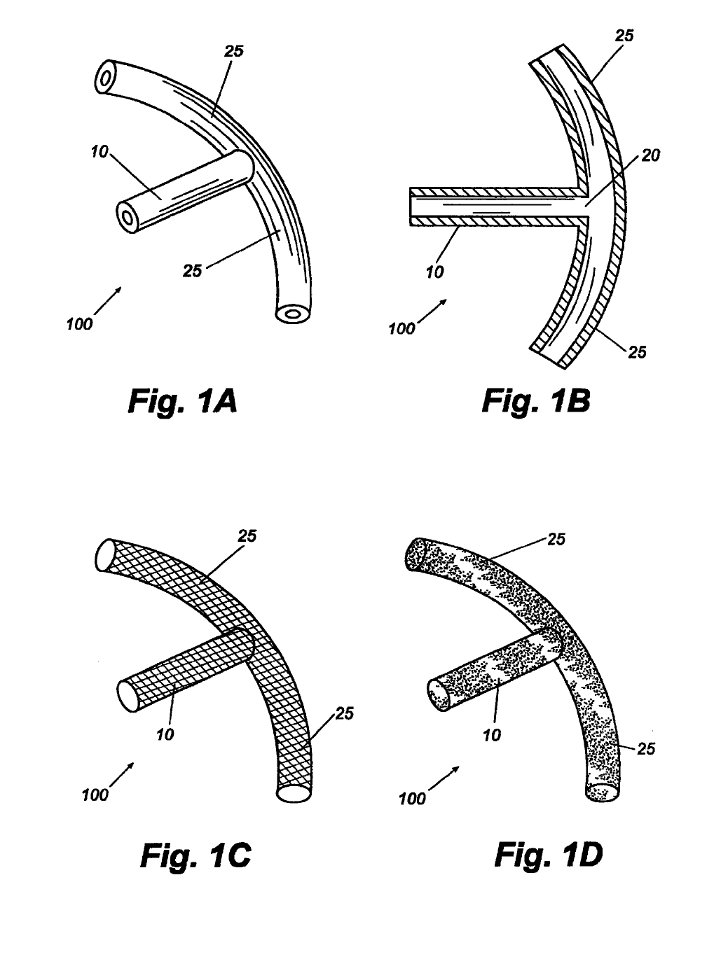

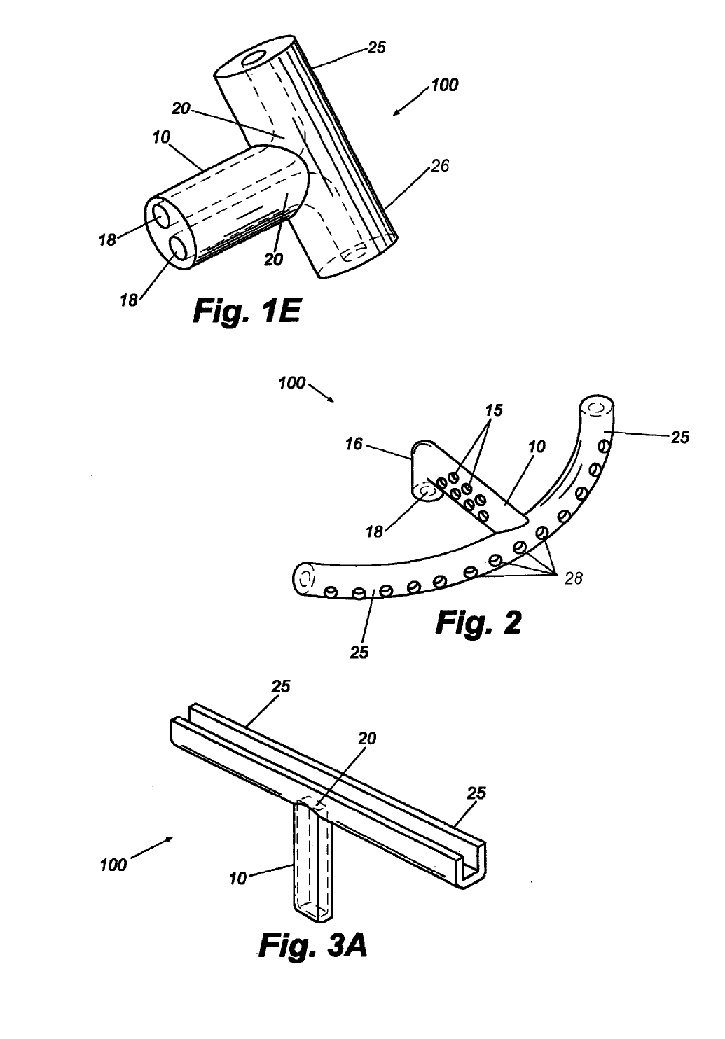

One embodiment of the present invention is illustrated in FIG. 1A, in which the shunt device 100 is shown in a side view. The shunt device 100 of this embodiment is comprised of two portions, a proximal portion 10 which joins a distal portion 25. The proximal portion 10 and distal portion 25 shown create an enclosed tubular channeling structure. The total length of the distal portion 25 may be between about 1.0 mm to 40 mm, preferably about 4 mm to 6 mm. The same embodiment is illustrated with phantom lines showing the internal fluid communication path in FIG. 1B. The lumen or channeling space defined by the walls of the proximal portion 10 and the distal portion(s) 25 are continuous at their junction at the distal portion portal 20.

Another embodiment is shown in FIG. 1C, in which the shunt device 100 is comprised of two luminal mesh elements, with a proximal portion 10 which joins a distal portion 25. Yet another embodiment is shown in FIG. 1D, in which the shunt device 100 is comprised of two solid, porous elements which may provide wick-like fluid communication therethrough, with a proximal portion 10 which joins a distal portion 25.

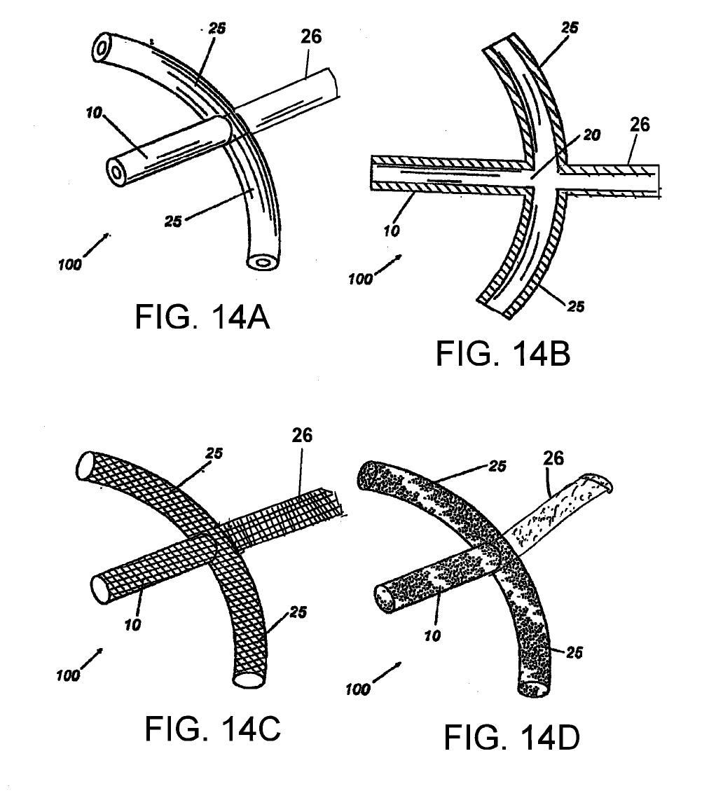

One embodiment is illustrated in FIG. 14A, in which the shunt device 100 is shown in a side view. The shunt device 100 of this embodiment is comprised of two portions, a proximal portion 10 which joins a distal portion 25 and distal portion 26. The proximal portion 10 and distal portions 25 and 26 shown create an enclosed tubular channeling structure. The total length of the distal portion 25 may be between about 1.0 mm to 40 mm, preferably about 4 mm to 6 mm. The same embodiment is illustrated in cross section showing the internal fluid communication path in FIG. 14B. The lumen or channeling space defined by the walls of the proximal portion 10 and the distal portion(s) 25 and 26 are continuous at their junction at the distal portion portal 20.

An additional embodiment is shown in FIG. 14C, in which the shunt device 100 is comprised of two luminal mesh elements, with a proximal portion 10 which joins a distal portion 25 and a second distal portion 26. A further embodiment is shown in FIG. 14D, in which the shunt device 100 is comprised of two solid, porous elements which may provide wick-like fluid communication therethrough, with a proximal portion 10 which joins a distal portion 25 and distal portion 26.

Another embodiment is shown in FIG. 1E, in which the shunt device 100 is comprised of a proximal portion 10 having two lumens therein terminating in proximal portion portals 18. Either or both of the distal portions 25 and 26 may be shaped and sized to be received within Schlemm's canal have separate lumens traversing therethrough from each of the distal portion portals 20.

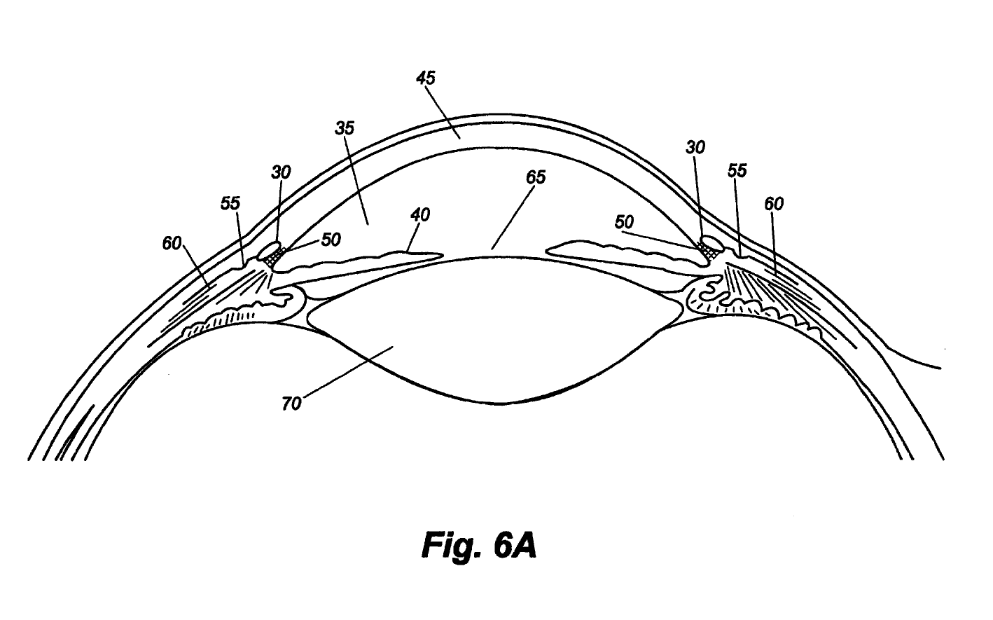

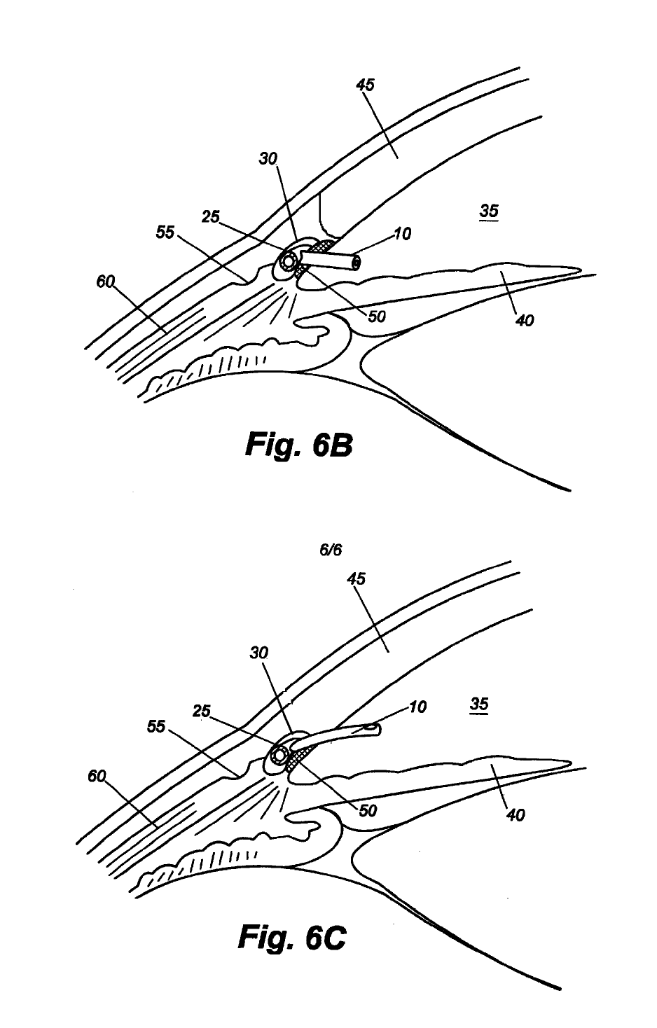

Other examples of embodiments are shown in FIGS. 2-5D. FIG. 2 shows an embodiment of the shunt in which the device 100 is tubular and fenestrated (15, 28) in its configuration, with an acute (<90) angle of junction between the proximal portion 10 and the plane defined by the distal portion 25. Such fenestrations (15, 28) may be placed along any portion of the device 100 to facilitate the passage of fluid therethrough, but are particularly directed towards the collecting channels of the eye. FIG. 2 further shows an additional embodiment of the shunt in which the terminal aspect 16 of the proximal portion is angulated toward the iris 40 with respect to the main axis of the proximal portion 10, with the portal 18 of the proximal portion directed toward from the iris 40. In other embodiments, such as that shown in FIG. 6C, the portal 18 of the proximal portion 16 is directed away from the iris 40.

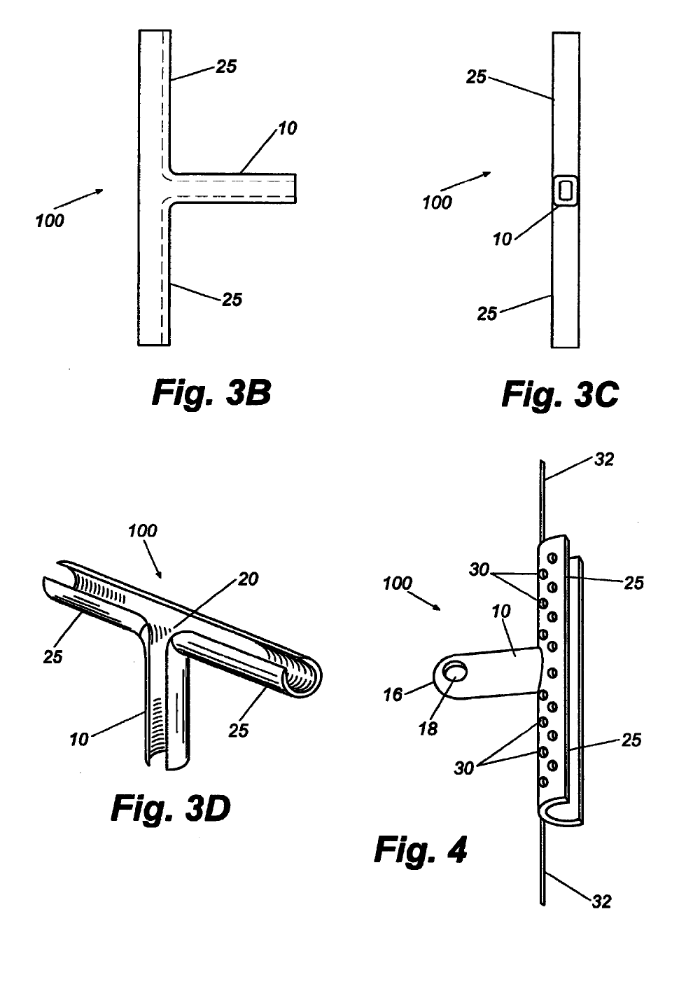

FIG. 3A shows an embodiment of the shunt in which a portion of the channeling device is enclosed and tubular in configuration at the junction of the proximal portion 10 and the distal portion 25, but where the distal portion 10 is a trough-like channel. The distal portion portal 20 is also shown. Any portion of the device 100 can be semi-tubular, open and trough-like, or a wick-like extension. Tubular channels can be round, ovoid, or any other enclosed geometry. Preferably the non-tubular trough-like aspects are oriented posteriorly on the outer wall of the canal to facilitate aqueous humor drainage to the collecting channels of the eye, as shown in FIG. 3A.

FIG. 3B shows an overhead view of the embodiment of the shunt of FIG. 3A, further detailing the relationship among the proximal portion 10 and the distal portion 25. The aqueous humor directing channel is shown in dashed lines. FIG. 3C shows a proximal view of the embodiment of the shunt of FIG. 3A, further detailing the relationship among the proximal portion 10 and the distal portion 25.

FIG. 3D shows another embodiment of the shunt in which the structure of the device 100 comprises an aqueous humor directing channel that is both open and curved in a continuous trough-like configuration along the proximal portion 10 and the distal portion 25. The distal portion portal 20 is also an open trough-like channel.

FIG. 4 shows another embodiment of the shunt with the addition of aqueous humor-wicking extensions 32 which are either continuous with, or attached to the terminal aspects of the distal portion 25. The wicking extensions 32 can be fashioned from a monofilament or braided polymer, such as proline, and preferably have a length of about 1.0 mm to about 16.0 mm. Furthermore, the proximal portion 10 is curved with a sealed, blunted tip 16 and contains a portal 18 in fluid communication with the lumen of the proximal portion and oriented to face away from the iris when the shunt device 100 is implanted in its intended anatomic position. The shunt device 100 can also help to maintain the patency of Schlemm's canal in a stenting fashion.

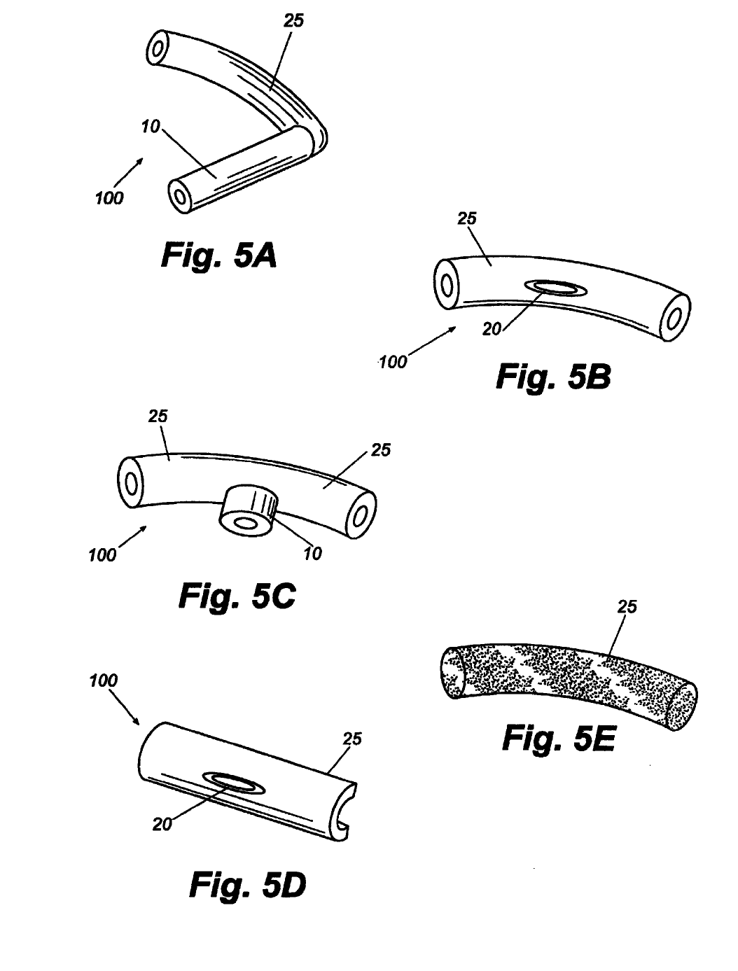

FIG. 5A shows another embodiment of the shunt in which the proximal portion 10 joins a single, curved distal portion 25 in a “V-shaped,” tubular configuration. The embodiment shown in FIG. 5A can also have a portal (not shown) in the distal portion 25 adjacent to the junction with the proximal portion 10 in order to facilitate bi-directional flow of fluid within the canal. Fenestrations and non-tubular, trough-like terminal openings are contemplated in all embodiments, and these fenestrations and openings may be round, ovoid, or other shapes as needed for optimum aqueous humor channeling function within the anatomic spaces involved.

FIG. 5B shows another embodiment of the shunt in which the body or device 100 comprises only a single, curved distal portion 25 which contains a distal portion portal 20 oriented towards the anterior chamber to allow egress of aqueous humor from the anterior chamber to Schlemm's canal. The body of this device can have a length of about 1.0 mm to about 40 mm, preferably about 6 mm. The external diameter of the device (or the distal portions of the device) can be about 0.1 mm to about 0.5 mm, preferably about 0.2 mm to about 0.3 mm, preferably about 0.23 mm to about 0.28 mm or about 0.26 mm.

FIG. 5C shows another embodiment of the shunt in which the device 100 comprises a bi-directional tubular distal portion 25 which is intersected by a proximal portion 10 which is short in length relative to the distal portion 25 and is directed towards the anterior chamber.

FIG. 5D shows still another embodiment of the shunt in which the device 100 comprises a bi-directional, trough-like, curved distal portion 25 for insertion into Schlemm's canal, which contains a distal portion portal 20 oriented to allow egress of aqueous humor from the anterior chamber, wherein the trough-like distal portion 25 is oriented to open toward the collecting channels to facilitate the egress of aqueous humor.

FIG. 5E shows another embodiment of the shunt in which the device 100 comprises a bi-directional, solid distal portion 25 for insertion into Schlemm's canal to facilitate the egress of aqueous humor from the canal to the collecting channels in a wicking capacity. The solid distal portion 25 can be porous or non-porous.

As the device is an implant, it can be fabricated from a material that will be compatible with the tissues and fluids with which it is in contact. The device may be constructed of biodegradable or non-biodegradable materials. It is preferable that the device not be absorbed, corroded, or otherwise structurally compromised during its in situ tenure. Moreover, it is equally important that the eye tissues and the aqueous remain non-detrimentally affected by the presence of the implanted device. A number of materials are available to meet the engineering and medical specifications for the shunts. In the exemplary embodiments, the shunt device 100 is constructed of a biologically inert, flexible material such as silicone or similar polymers. Alternate materials might include, but are not limited to, thin-walled Teflon®, polypropylene, other polymers or plastics, metals, or some combination of these materials. The shunt device 100 may be constructed as either porous or solid in alternate embodiments. The material can contain a therapeutic agent deliverable to the adjacent tissues.

In the embodiments shown in FIGS. 1A-4, the proximal portion 10 joins the distal portion(s) 25 at an angle sufficient to allow the placement of the proximal portion 15 within the anterior chamber of the eye when the distal portion 25 is oriented in the plane of Schlemm's canal. The proximal portion 10 is preferably of sufficient length, about 0.1 to about 3.0 mm or about 2.0 mm, to extend from its junction with the distal portion 25 in Schlemm's canal towards the adjacent space of the anterior chamber. While many geometries can be used for channeling aqueous humor, the diameter or width of the proximal portion 10 can be sized to yield an internal diameter of between about 0.1 and about 0.5 mm, preferably about 0.2 mm to about 0.3 mm for a tubular or curved shunt, or a comparable maximal width for a shunt with a multi-angular configuration. In other embodiments, the proximal portion is a non-luminal, non-trough-like wicking extension that provides an aqueous humor directing channel along the length thereof.

Because the nature of the iris 40 is such that it tends to comprise a plurality of rather flaccid fimbria of tissue, it is desirable to avoid said fimbria from being drawn into the lumen of an implant, thus occluding the shunt device. Therefore, the proximal portion 10 may contain a plurality of fenestrations to allow fluid ingress, arranged to prevent occlusion by the adjacent iris. Alternately, the proximal portion 10 may comprise only a proximal portion portal 18 in the form of a fenestration oriented anteriorly to provide continuous fluid egress between the anterior chamber of the eye and the directing channel of the shunt. Said fenestrations may be any functional size, and circular or non-circular in various embodiments. In addition, a porous structural material can assist in channeling aqueous humor, while minimizing the potential for intake of fimbria.

Furthermore, the proximal portion 10 may be positioned sufficiently remote from the iris 40 to prevent interference therewith, such as by traversing a more anterior aspect of the trabecular meshwork into the peripheral corneal tissue. In yet another possible embodiment, as shown in FIG. 6C, the device 100 may comprise a proximal portion 10 in which the terminal aspect of said proximal portion 10 is curved or angled toward the iris 40, and with a blunted, sealed tip 16 and a portal 18 oriented anteriorly to face away from the underlying iris 40. Such a configuration would tend to decrease the possibility of occlusion of the shunt device by the iris 40.

The device 100 may contain one or more unidirectional valves to prevent backflow into the anterior chamber from Schlemm's canal or the second anatomical space. The internal lumen for an enclosed portion of the device or the internal channel defined by the edges of an open portion of the device communicates directly with the inner lumen or channel of the distal portion at the proximal portion portal 20.

The distal portion 25 may have a pre-formed curve to approximate the 6.0 mm radius of Schlemm's canal in a human eye. Such a pre-formed curvature is not required when flexible material is used to construct the shunt device 100. The distal portion 25 may be of sufficient length to extend from the junction with the proximal portion 10 through any length of the entire circumference of Schlemm's canal. Embodiments having a distal portion 25 that extends in either direction within Schlemm's canal can extend in each direction about 1.0 mm to 20 mm, or about 3.0 mm. to permit circumferential placement through Schlemm's canal. The diameter or width of the distal portion 25 can be sized to yield an outer diameter of between about 0.1 and 0.5 mm, or about 0.3 mm, for a tubular or curved shunt, or a comparable maximal width for a shunt with a multi-angular configuration. The distal portion 25 may contain a plurality of fenestrations to allow fluid egress, arranged to prevent occlusion by the adjacent walls of Schlemm's canal. In other embodiments, the distal portion is a non-luminal, non-trough-like wicking extension that provides an aqueous humor directing channel along the length thereof.

In the exemplary embodiments, the shunt device may be either bi-directional, with the distal portion of the implant intersecting with the proximal portion in a “T-shaped” junction as shown in FIGS. 1A-1E, 2, 3A-3D, 4 and 5C, or uni-directional, with a “V-shaped” junction of the proximal and distal shunt portions, as shown in FIG. 5A. A bi-directional shunt device can have a distal portion that is threaded into opposing directions within Schlemm's canal. In the case of the unidirectional shunt, only the distal shunt portion is placed within Schlemm's canal. In these exemplary embodiments, “non-linear fluid communication” means that at least some portion of the shunt through which fluid passes is not in a straight line. Examples of non-linear shunts are the above described bi-directional “T” shapes, and the uni-directional “V” shapes, or shunts having two channel openings which are not in straight alignment with each other when implanted.

The surgical anatomy relevant to the present shunts is illustrated in FIG. 6A. Generally, FIG. 6A shows the anterior chamber 35, Schlemm's canal 30, the iris 40, cornea 45, trabecular meshwork 50, collecting channels 55, episcleral veins 60, pupil 65, and lens 70. FIG. 6B illustrates the surgical placement of the exemplary embodiment, with the relevant anatomic relationships. The device is designed so that placement of the distal portion 25 within Schlemm's canal 30 results in an orientation of the proximal portion 10 within the anterior chamber 35 within the angle defined by the iris 40 and the inner surface of the cornea 45. Therefore, if the plane defined by Schlemm's canal is defined as zero degrees, the proximal portion 10 can extend therefrom at an angle of between about +60 degrees towards the cornea 45 or −30 degrees toward the iris 40, more preferably in the range of 0 to +45 degrees. This range may vary in individuals having a slightly different location of Schlemm's canal 30 relative to the limbal angle of the anterior chamber 35.

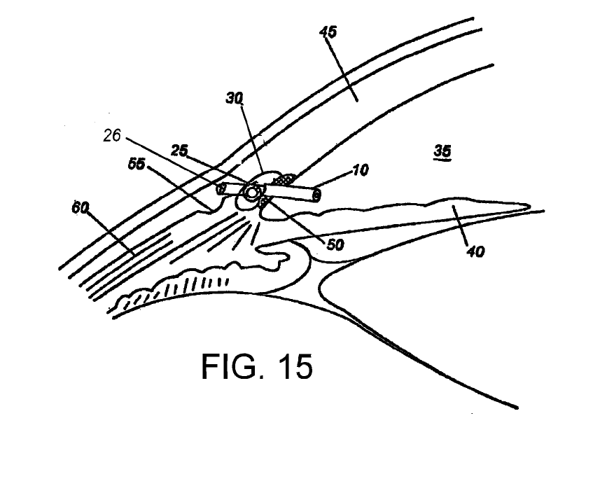

The surgical anatomy relevant to the present shunts is illustrated in FIG. 15. Generally, FIG. 15 shows the anterior chamber 35, Schlemm's canal 30, the iris 40, cornea 45, trabecular meshwork 50, collecting channels 55 and episcleral veins 60. This figure illustrates the surgical placement of the exemplary embodiment, with the relevant anatomic relationships. The device is designed so that placement of the distal portion 25 within Schlemm's canal 30 results in an orientation of the proximal portion 10 within the anterior chamber 35 within the angle defined by the iris 40 and the inner surface of the cornea 45. Therefore, if the plane defined by Schlemm's canal is defined as zero degrees, the proximal portion 10 can extend therefrom at an angle of between about +60 degrees towards the cornea 45 or −30 degrees toward the iris 40, more preferably in the range of 0 to +45 degrees. This range may vary in individuals having a slightly different location of Schlemm's canal 30 relative to the limbal angle of the anterior chamber 35. Similarly, the distal end 26 is positioned within the sclera.

In yet another embodiment, the shunt device 100 is configured with one distal portion 25 which is tubular to provide a shunting functionality and a plurality of proximal portions 10 which provide an anchoring function to stabilize the overall implant device, in addition to providing fluid communication from the anterior chamber to Schlemm's canal.

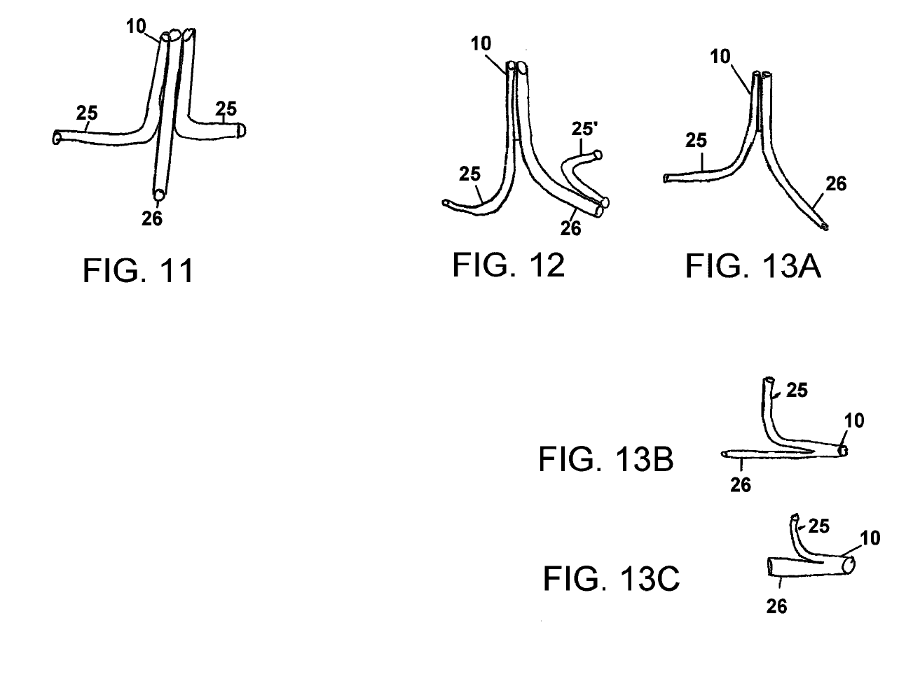

In another embodiment as shown in FIG. 11, the shunt may have more than two lumens entering the anterior chamber and a plurality of distal ends for placement into Schlemm's canal or the alternative anatomical spaces.

In yet another embodiment, as shown in FIG. 12, the shunt has dual lumens 25, 26 entering the anterior chamber, comprising a distal lumen 25 for positioning into Schlemm's canal and a second distal lumen 26 for drainage into the second anatomical space, wherein the second distal lumen 26 also has a secondary lumen 25′ for implantation into Schlemm's canal to facilitate further draining of the canal. In the particular embodiment depicted in FIG. 12, the shunt has three lumens, two of which extend from the anterior chamber into Schlemm's canal and the scleral space and a third which extends from the scleral space into Schlemm's canal to facilitate drainage of the canal into the sclera.

In another embodiment, as shown in FIG. 13A, the distal arms 25, 26 are of unequal length to facilitate drainage to different anatomical parts of the eye.

In another embodiment, as shown in FIG. 13B, the distal arms 25, 26 converge on a common lumen 10 which is inserted into the anterior chamber.

In another embodiment as shown in FIG. 13C, the distal arms 25, 26 are of unequal diameter or dimensions. Arm 26 going into the sclera can be plate like in its orientation.

Therefore, there is provided an aqueous humor shunt device to divert aqueous humor in an eye from the anterior chamber into Schlemm's canal, the shunt device comprising a distal portion having at least one terminal aspect sized and shaped to be received within a portion of Schlemm's canal and a proximal portion having at least one terminal aspect sized and shaped to be received within the anterior chamber of the eye, wherein the proximal portion has an anchor extending therefrom to maintain the position of the terminal aspect of the proximal portion within the anterior chamber of the eye, wherein device permits fluid communication from the proximal portion in the anterior chamber to the distal portion in Schlemm's canal. In other embodiments, such an anchor can extend from distal portions of the device to assist in stabilization of the implant within Schlemm's canal.

The multiple proximal portions or the anchor extension(s) from the distal or proximal portion (collectively referred to as the “anchor”) in the various embodiments described below and apparent to those of skill in the art in view of the present disclosure, provide multiple improvements for the shunt device. The anchor facilitates implantation and proper placement of the device, as the proximal portion can be advanced into the anterior chamber and then pulled back into place until it contacts the edge of the anterior chamber. As further described below, a shelf may be created by the surgical procedure for implantation that is designed to capture the anchor. This permits the surgeon to determine how much of the proximal portion is left extending into the anterior chamber. The anchor feature also allows the surgical alternative of first implanting the proximal portion into the anterior chamber, and then placing the distal portion(s) into Schlemm's canal. The anchor also serves to anchor the shunt device in the desired location within the anterior chamber and Schlemm's canal with minimal shifting during normal use.

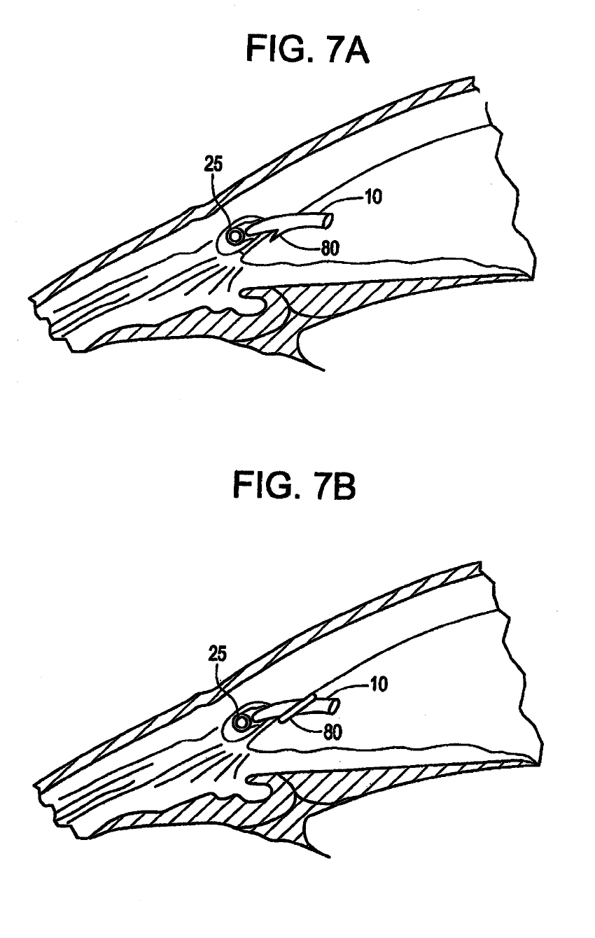

The anchor can be fabricated by a simple thickening of the material of construction of the shunt, e.g. silicone, at the desired site on the proximal portion, or can be made of another material attached thereto. Additionally, the anchor can be fabricated by removal of excess material. The anchor can extend from the proximal portion in virtually any functional shape, such as in a rounded or barbed fashion. FIG. 7A is a cross-sectional illustration showing the anatomic relationships of the surgical placement of an exemplary embodiment showing the proximal portion 10 of the device and a barb-shaped anchor 80 extending toward the iris. FIG. 7B is a cross-sectional illustration showing the anatomic relationships of the surgical placement of another exemplary embodiment showing the proximal portion 10 of the device having an annular or circumferential anchor 80 thereon.

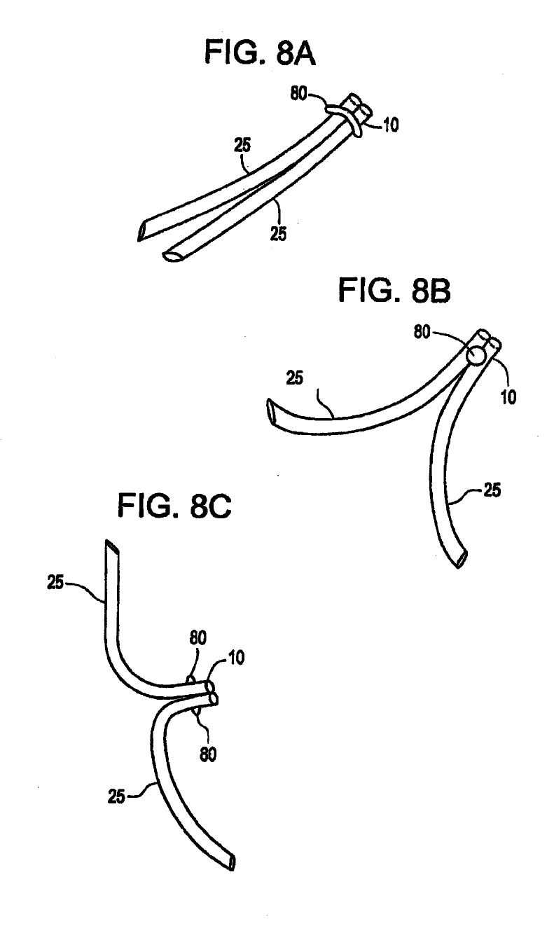

Therefore, the anchor can extend circumferentially around the proximal portion, or only in one or more directions therefrom. FIG. 8A shows one embodiment of the device having a bi-directional distal portion 25 and an anchor 80 on the proximal portion 10 extending circumferentially thereon. FIG. 8B shows another embodiment of the device having a bi-directional distal portion 25 and an anchor 80 on the proximal portion 10 extending medially toward the location of the iris when implanted. FIG. 8C shows another embodiment of the device having a bi-directional distal portion 25 and an anchor 80 on the proximal portion 10 extending laterally on each side of the device when implanted. The invention contemplates many other configurations of the anchor, including a plurality of teeth extending from the proximal portion.

The device may also be provided with an anchor for placement adjacent the exterior surface of the anterior chamber to assist in surgical placement and securing the device, with or without a corresponding anchor adjacent the interior surface of the anterior chamber. Thus, a potential configuration to stabilize the implant is a device having anchors for positioning inside the anterior chamber and inside Schlemm's canal to secure the device about the trabecular meshwork between the anterior chamber and Schlemm's canal.

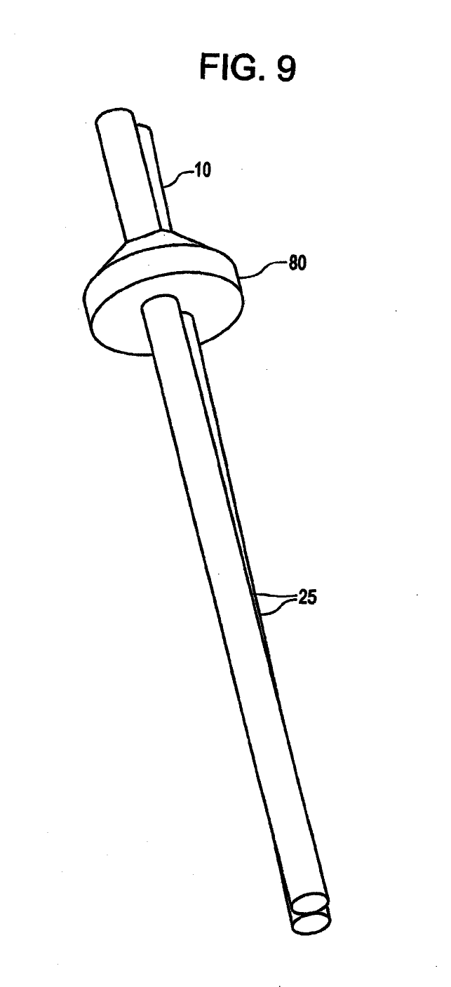

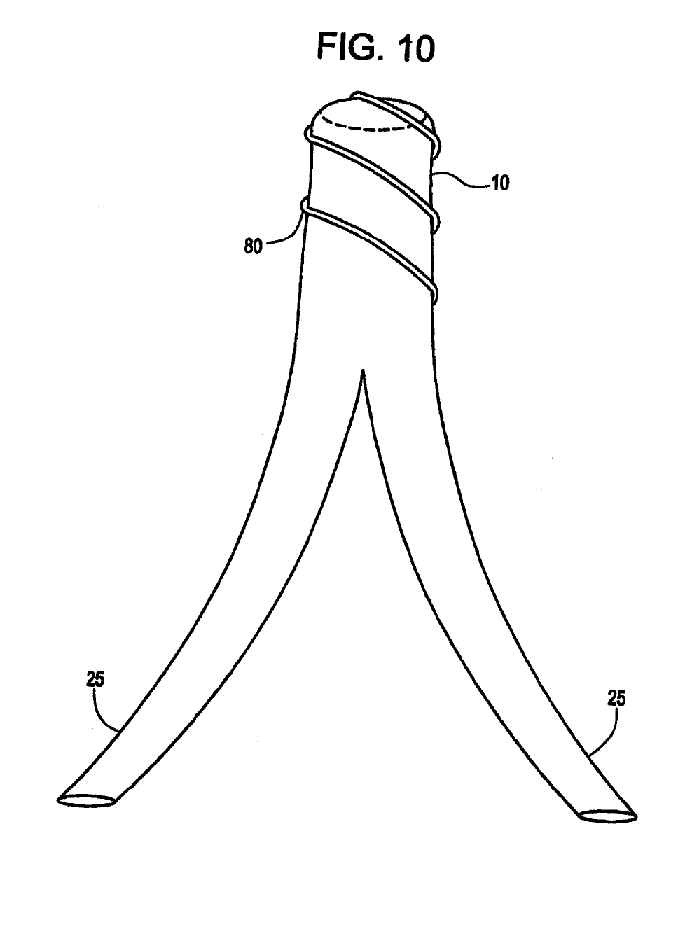

It is understood that the anchor can extend in any direction in any shape and size which facilitates implantation or anchoring of the device. For example, FIG. 9 shows another embodiment having a bi-directional distal portion 25 and an anchor 80 on the proximal portion 10 extending circumferentially thereon in a barbed or cone shape to facilitate introduction into the anterior chamber and to inhibit removal therefrom. Furthermore, the end of the proximal portion can be cut at an angle, rather than blunted or square cut, in order to facilitate introduction through the wall of the anterior chamber. The angled shape of the tip of the proximal portion allows the proximal portal to have a larger surface area to facilitate the flow of aqueous. The device preferably is at least capable of permitting the flow of aqueous humor at the estimated normal production rate of about 2.5 microliters per minute.

FIG. 10 shows yet another embodiment of the device in which the proximal end comprises a larger single proximal lumen 10 which branches to form a pair of distal lumens 25 for insertion into Schlemm's canal. The proximal end is preferably tapered and contains screw threads 80 such that the device can be screwed into the anterior chamber and anchored therein by means of the threads and the distal ends inserted in Schlemm's canal. This embodiment would, in some instances, simplify insertion by eliminating the need to make an incision into the anterior chamber.

The anchor, as well as optionally the remainder of the device, can be constructed on a textured, grooved or porous material in order to facilitate the growth of cells, such as fibroblasts, to stabilize the implant from movement. Preferably, the extreme tips of the proximal and distal ends of the device are produced to avoid the attraction of new tissues, such as fibroblasts, which may grow at the surgical site and impede the flow of aqueous therethrough. Therefore, the proximal portion of the device can be produced to extend beyond the entrance into the anterior chamber by 0.1 to 3 mm, or preferably about 0.5 mm. As discussed above, the angled tip of the proximal portal will create a range of lengths along the proximal portion extending into the anterior chamber.

The distal portion(s), preferably, similarly extends beyond the site of surgery and subsequent fibroblast proliferation. Therefore, the distal portion(s) can have a length of approximately 4 mm to 6 mm, again taking into consideration variability for angled extremities. The single or dual lumen shunt devices can be manufactured by conventional molding or extrusion techniques. In the case of extrusion production, single lumens can be subsequently partially joined together to form dual or plural lumen devices. Alternatively, the lumens can be co-extruded as a plurality of lumens and the individual lumens can be partially separated to define distal portions extendable in separate directions. In this manner, devices having two, three, four or more lumens can be fabricated in a minimal of pieces. It is preferable that such devices be constructed such that they will not kink when wrapped around a 0.25 mm object. The silicone tubes can be any geometric shape and that the lumens can be cut to form troughs, plates and/or fenestrated anywhere along their length to produce desired rates and directions of flow.

Optionally, the device may also include one or more visible markings on the device to assist in proper placement in the anterior chamber or Schlemm's canal. Markings on the distal ends could be used to confirm the distal ends are properly inserted in Schlemm's canal and markings on the proximal end would avoid over or under insertion into the anterior chamber.

Optionally, the device may be selectively coated or permeated with therapeutic agents as desired. For example, where in-growth is desired for stability, certain growth factors may be present, whereas at the terminal portals where obstructions are to be avoided, certain antifibrotic agents may be present, such as 5-fluourouracil or mitomycin. The device may be more generally provided with coatings that are antibiotic, anti-inflammatory, or carboxylic anhydrase inhibitors. Agents that facilitate the degradation of collagen within the trabecular meshwork can also be employed.

The present device, unlike other filtering procedures, does not leave any external openings in the eye for bacteria to enter. Instead, the implants are wholly implanted with in the eye and drain through natural pathways either through Schlemm's canal, the sclera or the uveoscleral routes. Regulation of flow occurs through natural resistance to flow inherent in the tissues in which the shunt device is implanted, e.g., the walls of Schlemm's canal, the membranes defining the uveoscleral spaces and the tissue of the sclera. By using this natural flow resistance no other flow regulating means is necessary for the device. When using devices, the device is sized for flow rate by changing the inside diameter of the tube to increase or decrease flow as desired. Such sizing is of course limited by the dimensions of the tissue spaces into which the portion of the device is implanted.

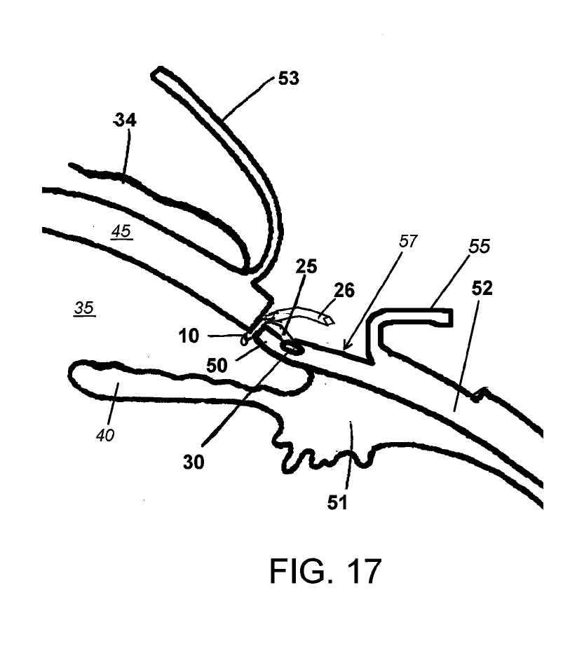

The present invention provides methods for the implantation and use of the shunt devices. One surgical procedure that may be used to insert the device is illustrated in FIG. 17 and involves an approach through a conjunctival flap 34. A partial thickness scleral flap 53 is then created and dissected half-thickness into clear cornea 45. Continue the dissection along a more shallow plane to create a corneo-scleral shelf 57 over the trabecular meshwork 50. The posterior aspect of Schlemm's canal 30 is identified and the canal 30 is entered posteriorly. Schlemm's canal 30 and/or the anterior chamber 35 may be expanded and lubricated by injection of a viscoelastic and/or a mitotic agent. Suitable viscoelastic compositions and devices and methods for their injection into the eye are disclosed in U.S. Pat. No. 5,360,399 which is incorporated herein by reference. When using viscoelastic compositions, care should be taken to avoid over-expanding and rupturing Schlemm's canal 30. The proximal portion 10 of the shunt is then inserted through the inner wall of Schlemm's canal 30 and trabecular meshwork 50 into the anterior chamber 35 within the angle between the iris 40 and the cornea 45. In some cases, an incision may be needed from Schlemm's canal 30 through the trabecular meshwork 50 into the anterior chamber 35 to facilitate passage of the proximal portion 10 therethrough. One arm of the distal portion 25 of the shunt device is grasped and threaded into Schlemm's canal 30 and the opening closed with tissue 55 from the scleral shelf 57. The second distal portion 26 of the shunt device is then placed in the opening made by the initial scleral flap. A suture may be used at the surgeon's discretion to anchor arm within the sclera 52. The scleral flap 53 and conjunctival wound 34 are closed in a conventional manner. Drainage into the sclera 52 will create a bleb. Alternatively, as mentioned above for FIG. 16, a distal end of the device portion 26 may be inserted into the potential space between the ciliary body 51 and the sclera 52 to drain into the uveoscleral route.

The following procedure may also be followed for the insertion of a shunt within Schlemm's canal and the sclera:

1) Obtain general or local anesthesia. Preferably with either a retro-bulbar or peribulbar injection of an anesthetic agent (lidocaine, bupivacaine, etc.).

2) Scrub the periocular region with a surgically acceptable antiseptic such as povodine iodine solution. Place a lid speculum.

3) Make a formix-based conjunctival incision at the limbus. Ensure hemostasis with either bipolar cautery or diathermy.

4) Make a 3-4 mm×3-4 mm scleral flap, extending to a depth within approximately 100 of the choroid.

5) Dissect the flap anteriorly to unroof the outer wall of Schlemm's canal.

6) Continue the dissection along a more shallow plane to create a corneo-scleral shelf over the trabecular meshwork. At surgeon's discretion, place a stay suture through the scleral flap to hold it in position.

7) At surgeon's discretion, dilate the opening to Schlemm's canal on both sides of the flap using a viscocanalostomy cannula and a viscoelastic agent (e.g., hyaluronate or hyaluronate/chondroitin sulfate).

8) Make a paracentesis at the limbus distal to the surgical site.

9) At surgeon's discretion, inject a viscoelastic agent and a miotic (carbachol or acetylcholine) into the anterior chamber to deepen the area.

10) Remove the shunt from its case. Insert one of the distal portions of the shunt into the canal on one side or the other.

11) Enter the anterior chamber along the corneo-scleral shelf using a keratome blade or a 21 gauge needle.

12) Insert the proximal portion of the tube into the anterior chamber.

13) Anchor the second distal portion onto the scleral shelf using 10-0 nylon sutures.

14) Close the scleral flap with interrupted 10-0 nylon sutures. Initially, place one suture at the base and one each along the two sides. Bury the suture knots.

16) Deepen the anterior chamber with balanced salt solution through the paracentesis.

17) Test the scleral flap with a cellulose sponge. If there is leakage, place additional 10-0 nylon sutures to achieve a watertight closure.

18) Close the conjunctiva with appropriately sized absorbable sutures.

19) Dress the eye with subconjunctival and/or topical broad-spectrum antibiotic and corticosteroid.

20) Place a protective shield over the eye and tape the shield in place.

For patients that have had a prior device of the present design implanted into both arms of Schlemm's canal, a variation on the above procedure can be utilized to move one of the arms to the sclera.

1) Obtain general or local anesthesia, preferably with either a retro-bulbar or peribulbar injection of an anesthetic agent (lidocaine, bupivacaine, etc.).

2) Scrub the periocular region with a surgically acceptable antiseptic such as povodine iodine solution. Place a lid speculum.

3) Make a formix-based conjunctival incision at the limbus. Ensure hemostasis with either bipolar cautery or diathermy.

4) Make a 3-4 mm×3-4 mm scleral flap, extending to a depth within approximately 100 of the choroid.

5) Dissect the flap anteriorly to unroof the outer wall of Schlemm's canal.

6) Remove one of the distal arms of the shunt from within Schlemm's canal.

7) Anchor the removed portion onto the scleral shelf using 10-0 nylon sutures.

8) Close the scleral flap with interrupted 10-0 nylon sutures. Initially, place one suture at the base and one each along the two sides. Bury the suture knots.

9) Deepen the anterior chamber with balanced salt solution through the paracentesis.

10) Test the scleral flap with a cellulose sponge. If there is leakage, place additional 10-0 nylon sutures to achieve a watertight closure.

11) Close the conjunctiva with appropriately sized absorbable sutures.

12) Dress the eye with subconjunctival and/or topical broad-spectrum antibiotic and corticosteroid.

13) Place a protective shield over the eye and tape the shield in place.

The above procedure was performed in three patients whose pressures remained elevated after implantation of the device wholly into Schlemm's canal. Table 1 shows the pressures before and after the surgery. All pressures are in millimeters of mercury (mm Hg).

TABLE 1
Follow-up dates and intra-ocular pressure (mmHg) data of patients receiving an
alternative surgery after unsuccessfully completing the Phase 2 and Phase 3
clinical studies with the Eyepass ® Glaucoma Implant.

| | | | | | | | | |
|---|---|---|---|---|---|---|---|---|
| | Most recent | IOP ± 1 | IOP ± | IOP ± | IOP ± | IOP ± | IOP ± | IOP ± |
| | IOP prior to | Day | 1 Wk | 1 Mo | 3 Mos | 6 Mos | 9 Mos | 1 Yr |
| Patients^{o} | Surgery | F/U | F/U | F/U | F/U | F/U | F/U | F/U |

| | | | | | | | |
|---|---|---|---|---|---|---|---|
| EJC #0204 | 27 on 4 meds | 3 | 12 | 16 | 19 | 15 on 2 | 21 on 2 |
| | | | | | | meds | meds |
| ETT #0208 | 23 on 3 meds | 18 | 11 | 7 | 8 | 8 | 8 on |
| | | | | | | | no |
| | | | | | | | meds |
| CEH #0501 | 25 | 2 | 12 | 13 | 14 | 18 on | |
| | 3 meds | | | | | no | |
| | | | | | | meds | |

No complications were observed in any of the patients.

Alternatively the distal arm of the shunt can be terminated only within the sclera. In this instance the implantation procedure is simplified because there is no need to locate Schlemm's canal. A small conjunctival incision would be made about 2.5 mm back from the limbus and a tunnel created through scleral into the anterior chamber. The proximal end of the shunt would then be inserted directly into the anterior chamber. An additional tunnel would be created posteriorly to create a path for the distal end of the shunt. The shunt preferably is positioned (trimmed if necessary) so just the distal end of the shunt is peeking out from the back end of the tunnel. A second small incision is made away from the distal end of the shunt in an area where a drainage bleb is desired. An expanding blunt dissector would be passed through the subconjunctival space to create the bleb-space. Once opened a sponge containing Mitomycin-C would be inserted into that space and removed. Once the Mitomycin-C has been applied, the two incisions would be closed with 10-0 sutures.

This procedure would shunt aqueous fluid from the anterior chamber to a bleb 5 mm from the limbus. The procedure would have many advantages over other known procedures. First, the incisions are tiny and there is no need to remove any tissue. Second, no iridectomy is required. Third, there is no need to find Schlemm's canal. Fourth, the conjunctiva is much easier to work with posteriorly. Fifth, the risk of infection and bleb irritation because the shunt is implanted in the sclera is much less. Sixth, the process may require about 10-15 minutes per eye.

One of skill in the art will appreciate that there are many potential embodiments of the devices disclosed herein and that new materials may enable the creation of additional variations which do not deviate from the spirit of the invention. This includes devices which combine tubes, troughs, wicks in a single device. Similarly, the surgical process may be simplified by the development of new tools and techniques which likewise do not deviate from the spirit of the invention. It will also be appreciated that multiple devices can be implanted in a single eye.

What is claimed is:

1. An ocular implant for reducing intraocular pressure within an eye, comprising:
a proximal portion configured to extend into an anterior chamber of the eye when the implant is finally positioned within the eye;
a first distal arm configured to extend from the proximal portion into a Schlemm's canal when the implant is finally positioned within the eye;
a second distal arm configured to extend from the proximal portion into a suprachoroidal space of the eye when the implant is finally positioned within the eye;
a first aqueous humor directing channel extending from the proximal portion to the first distal arm, the first aqueous humor directing channel configured to drain aqueous humor from the anterior chamber to the Schlemm's canal; and
a second aqueous humor directing channel extending from the proximal portion to the second distal arm, the second aqueous humor directing channel configured to drain aqueous humor from the anterior chamber to the suprachoroidal space.

2. The ocular implant of claim 1, wherein the proximal portion, the first distal arm and the second distal arm comprise a single, monolithic piece.

3. The ocular implant of claim 1, wherein the first aqueous humor directing channel and the second aqueous humor directing channel comprise lumens.

4. The ocular implant of claim 1, wherein at least one of the first aqueous humor directing channel and the second aqueous humor directing channel comprises a trough-like channel.

5. The ocular implant of claim 1, wherein the first distal arm and the second distal arm are of unequal length.

6. The ocular implant of claim 1, wherein the first distal arm and the second distal arm are substantially tubular.

7. The ocular implant of claim 1, wherein the second distal arm is plate-like.

8. An ocular implant for reducing intraocular pressure within an eye, comprising:
a proximal portion extending into an anterior chamber of the eye upon implantation;
a first distal portion extending into Schlemm's canal upon implantation; and
a second distal portion extending into a second anatomical space of the eye upon implantation.

9. The ocular implant as in claim 8, wherein the proximal portion, the first distal arm and the second distal arm comprise a single, monolithic piece.

10. The ocular implant as in claim 8, wherein the second anatomical space is a suprachoroidal space.

11. The ocular implant as in claim 8, wherein the second anatomical space is a subconjunctival space.

12. The ocular implant as in claim 8, further comprising a first aqueous humor directing channel extending between the proximal portion and the first distal portion and a second aqueous humor directing channel extending between the proximal portion and the second distal portion.

13. The ocular implant as in claim 8, further comprising a first aqueous humor directing channel extending between the proximal portion and the first distal portion and a second aqueous humor directing channel extending between the first distal portion and the second distal portion.

14. The ocular implant as in claim 12, wherein the first aqueous humor directing channel comprises one or more lumens.

15. The ocular implant as in claim 12, wherein the second aqueous humor directing channel comprises one or more lumens.

16. The ocular implant as in claim 12, wherein the first aqueous humor directing channel comprises a trough-like channel.

17. The ocular implant as in claim 10, wherein the second distal portion is sized and shaped to fit within the suprachoroidal space.

18. The ocular implant as in claim 10, wherein the second distal portion extending to the suprachoroidal space is plate-like in its orientation.

19. An aqueous shunt device for reducing intraocular pressure within an eye, comprising:
at least one proximal end sized and shaped to be positioned within an anterior chamber of the eye;
a first distal end sized and shaped to be positioned within a first physiologic outflow route of the eye; and
a second distal end sized and shaped to be positioned within a second physiologic outflow route of the eye that is not connected to the first physiologic outflow route,
wherein the shunt device comprises a single, monolithic device.

20. The aqueous shunt device of claim 19, wherein the first physiologic outflow route is a canalicular route and wherein the second physiologic outflow route is a uveoscleral route.
